(19) United States
(12) Patent Application Publication
(10) Pub. No.: US 2026/0085307 A1
(43) Pub. Date: Mar. 26, 2026

(54) GENOME-WIDE INSULATOR SCREENING SYSTEM AND USE

(71) Applicant: Agricultural Genomics Institute at Shenzhen, Chinese Academy of Agricultural Sciences (Guangdong, Shenzhen (CN)

(72) Inventors: Lei HUANG, Shenzhen (CN); Yuwen LIU, Shenzhen (CN); Xiusheng ZHU, Shenzhen (CN)

(21) Appl. No.: 19/021,326

(22) Filed: Jan. 15, 2025

(30) Foreign Application Priority Data
Sep. 24, 2024 (CN) 202411329018.6

(51) Int. Cl. C12N 15/10; C12N 15/66; C12Q 1/6806; C12Q 1/6809; C12Q 1/6855; C12Q 1/686; C12Q 1/6869

(52) U.S. Cl. CPC C12N 15/1082; C12N 15/66; C12Q 1/6806; C12Q 1/6809; C12Q 1/6855; C12Q 1/686; C12Q 1/6869; C12N 2830/50

(57) ABSTRACT

The present disclosure discloses a genome-wide insulator screening system. The system comprises an MAI-seq-experiment vector and an MAI-seq-control vector; core gene elements in the MAI-seq-experiment vector are arranged in following order: a weak promoter, a marker target gene, an insertion site for a sequence to be screened, an enhancer and a poly A site; and core gene elements in the MAI-seq-control vector are arranged in following order: a weak promoter, a marker target gene, an enhancer, an insertion site for the sequence to be screened and a poly A site. This genome-wide insulator screening system, primarily composed of these two vectors, exhibits high sensitivity and can be applied to the screening of genomic insulators in any species. Furthermore, it effectively eliminates the influence of silencers, ensuring high screening accuracy. This system provides important technical support for constructing insulator maps and understanding the characteristics and mechanisms of action of insulators.

### SEQUENCE LISTING

This application contains a sequence listing, the copy of which is named SEQ GENOME-WIDE INSULATOR SCREENING SYSTEM AND USE THEREOF.xml, created on Jan. 6, 2025, and has a size of 19,797 bytes.

### TECHNICAL FIELD

The present disclosure relates to the field of genetic engineering, particularly to a genome-wide insulator screening system and use thereof.

### BACKGROUND

Cis-acting elements are non-coding DNA regions that regulate gene expression by binding to transcription factors, thereby controlling the precise initiation of gene transcription and transcriptional efficiency. These cis-regulatory elements mainly include promoters, enhancers, silencers, and the relatively less-studied insulators. Insulators were initially discovered in Drosophila cells, and their molecular mechanisms have since been elucidated. When located between a gene and an enhancer, insulators can prevent the enhancer from exerting its effect on the target gene. Additionally, insulators can block the repressive effects of heterochromatin near active genes on those active genes. Insulators play a crucial role in the structure and function of the mammalian genome, but little is known about how insulators exert their functional specificity, primarily due to the lack of effective methods for insulator identification.

### SUMMARY

The objective of the present disclosure is to provide a genome-wide insulator screening system and use thereof, which can be efficiently used for screening genomic insulators in any species. The construction method and use of this screening system provide important support for constructing insulator maps and understanding the characteristics and mechanisms of action of insulators.

In the first aspect, the present disclosure provides a genome-wide insulator screening system. The system comprises an MAI-seq-experiment vector and an MAI-seq-control vector; core gene elements in the MAI-seq-experiment vector are arranged in following order: a weak promoter, a marker target gene, an insertion site for a sequence to be screened, an enhancer and a poly A site; and core gene elements in the MAI-seq-control vector are arranged in following order: a weak promoter, a marker target gene, an enhancer, an insertion site for the sequence to be screened and a poly A site. Thus, this genome-wide insulator screening system, primarily composed of these two vectors, exhibits high sensitivity and can be applied to the screening of genomic insulators in any species. Furthermore, it effectively eliminates the influence of silencers, ensuring high screening accuracy. This system provides important technical support for constructing insulator maps and understanding the characteristics and mechanisms of action of insulators.

In some embodiments, the insertion site for the sequence to be screened contains homologous arm sequences that are arranged to undergo homologous recombination, and after two ends of a DNA sequence to be screened are added to the homologous arm sequences, homologous recombination occurs with either the MAI-seq-experiment vector or the MAI-seq-control vector.

In some embodiments, a nucleotide sequence of the MAI-seq-experiment vector is as shown in SEQ ID NO: 1, and a nucleotide sequence of the MAI-seq-control vector is as shown in SEQ ID NO:2.

In the second aspect, the present disclosure provides a use of the aforementioned genome-wide insulator screening system in screening for genomic insulators. This use involves fragmenting the gene sequence to be tested, adding Illumina® sequencing adapters, and performing PCR amplification using primers containing homology arms that can undergo homologous recombination with the screening system vectors. This process adds homology arm sequences to both ends of the DNA fragments of the gene sequence to be tested, enabling them to undergo homologous recombination and insertion at specified locations in the screening system vectors. Leveraging the position-dependent manner in which insulators function, this use not only facilitates the screening of insulators but also effectively eliminates the influence of silencers. The system and use method are simple, efficient, highly sensitive, and applicable to any species.

In some embodiments, the use comprises following steps:
S1: fragmenting a gene sequence to be tested, ligating the fragmented gene segments with Illumina® sequencing adapters, and performing PCR amplification using primers as shown in SEQ ID NO:5 and SEQ ID NO:6;
S2: homologously recombining PCR products from S1 into the MAI-seq-experiment vector and the MAI-seq-control vector to construct homologous recombination plasmids;
S3: transforming the homologous recombination plasmids constructed in S2 and extracting the plasmids to obtain an Input library for the MAI-seq-experiment vector and an Input library for the MAI-seq-control vector, respectively, and performing high-throughput sequencing on the Input libraries;
S4: transfecting cells with the Input libraries of the MAI-seq-experiment vector and the MAI-seq-control vector constructed in S3, collecting the cells, performing reverse transcription amplification, collecting amplified products to prepare an Output library for the MAI-seq-experiment vector and an Output library for the MAI-seq-control vector, respectively, and performing high-throughput sequencing on the Output libraries;
S5: by comparing a sequencing data of the Input and Output libraries of the MAI-seq-experiment vector, screening out sequences with reduced transcription abundance in the Output library compared to the Input library;
S6: by comparing a sequencing data of the Input and Output libraries of the MAI-seq-control vector, screening out sequences with reduced transcription abundance in the Output library compared to the Input library; and
S7: removing from the sequences screened out in S5 those that are duplicated in the sequences screened out in S6, with the remaining sequences being the screened insulator sequences.

In the third aspect, the present disclosure provides a use of the aforementioned genome-wide insulator screening system in validating genomic insulators. Thereby, this use merely requires amplifying the sequence to be validated by PCR using primers containing homology arms that can undergo homologous recombination with the MAI-seq-experiment vector in the genome-wide insulator screening system. The amplified sequence to be validated, containing homology arms, is then homologously recombined with the MAI-seq-experiment vector and inserted into the specified location. By detecting changes in the expression level of the marker target gene, it is possible to validate whether the sequence is an insulator sequence.

In some embodiments, the use comprises following steps:
S1: inserting the sequence to be validated into the MAI-seq-experiment vector to construct a validation plasmid;
S2: using the MAI-seq-experiment vector without any sequence inserted as a blank control plasmid; and
S3: transfecting cells with the blank control plasmid and the validation plasmid separately, collecting cells 48 hours post-transfection to extract RNA, and utilizing qPCR to detect changes in the expression level of the marker target gene. If the expression level of the marker target gene in the validation plasmid group decreases, it proves that the sequence to be validated is an insulator; otherwise, it is not.

In the fourth aspect, the present disclosure provides a use of the aforementioned genome-wide insulator screening system in a preparation of a kit or a product for screening or validating genomic insulators.

In the fifth aspect, the present disclosure provides an insulator validation vector. The insulator validation vector is the MAI-seq-experiment vector with its nucleotide sequence shown in SEQ ID NO:1. Thereby, using this MAI-seq-experiment vector, one only needs to insert the sequence to be validated into a designated position of the vector via homologous recombination, and then by comparing the abundance of the marker target gene expression, it is possible to preliminarily determine whether the sequence to be validated is an insulator in a simple, rapid, efficient, and convenient manner.

In the sixth aspect, the present disclosure provides a use of the aforementioned insulator validation vector in the screening and validation of insulators.

The beneficial effects of the present disclosure are as follows:

1. The genome-wide insulator screening system disclosed in the present disclosure primarily consists of two vectors, exhibiting high sensitivity and applicability for screening genomic insulators in any species. Moreover, it effectively eliminates the influence of silencers, ensuring high screening accuracy. This system provides crucial technical support for constructing insulator maps and understanding the characteristics and mechanisms of action of insulators.

2. The present disclosure discloses a use of the genome-wide insulator screening system in screening for genomic insulators and a use method. This method involves fragmenting the gene sequence to be tested and adding Illumina® sequencing adapters. Subsequently, PCR amplification is performed using primers containing homology arms that can recombine homologously with the vectors in the genome-wide insulator screening system. This results in the addition of homology arm sequences to both ends of the DNA fragments of the gene sequence to be tested. These homology arms enable the DNA fragments of the gene sequence to be tested to recombine homologously with the vector and be inserted into a specified position. By leveraging the position-dependent functionality of insulators, the screening of insulators is achieved, effectively eliminating the influence of silencers. This system and use method are simple, efficient, highly sensitive, and applicable to any species.

3. The present disclosure also discloses a use of the genome-wide insulator screening system in validating genomic insulators and a use method. This method merely requires amplifying the sequence to be validated by PCR using primers containing homology arms that can undergo homologous recombination with the MAI-seq-experiment vector in the genome-wide insulator screening system. The amplified sequence to be validated, containing homology arms, is then homologously recombined with the MAI-seq-experiment vector and inserted into the specified location. By detecting changes in the expression level of the marker target gene, it is possible to validate whether the sequence is an insulator sequence.

4. Furthermore, the present disclosure discloses an insulator validation vector. Using this MAI-seq-experiment vector, one only needs to insert the sequence to be validated into a designated position of the vector via homologous recombination, and then by comparing the abundance of the marker target gene expression, it is possible to determine whether the sequence is an insulator in a simple, rapid, efficient, and convenient manner.

### BRIEF DESCRIPTION OF THE DRAWINGS

FIG. 1 shows the structural diagram of the MAI-seq-experiment vector;

FIG. 2 shows the structural diagram of the MAI-seq-control vector;

FIG. 3 shows a schematic diagram of the main gene sequence of the MAI-seq-experiment vector, where Mini Promoter represents the weak promoter Mini Promoter sequence, Green Fluorescent Protein (GFP) stands for the marker gene GFP sequence, Insulator serves as the insertion site for the sequence to be screened, SV40 Enhancer denotes the enhancer SV40 Enhancer sequence, and poly (A) signal signifies the poly A site sequence;

FIG. 4 shows a schematic diagram of the main gene sequence of the MAI-seq-control vector, where Mini Promoter represents the weak promoter Mini Promoter sequence, GFP stands for the marker gene GFP sequence, SV40 Enhancer denotes the enhancer SV40 Enhancer sequence, Insulator serves as the insertion site for the sequence to be screened, and poly (A) signal signifies the poly A site sequence;

FIG. 5 shows the results of validating the insulation sequence screened by the MAI-seq-experiment vector: ** indicates a highly significant difference compared to the control group, p<0.01; * indicates a significant difference compared to the control group, p<0.05.

### DETAILED DESCRIPTION OF THE EMBODIMENTS

The present disclosure is further described in detail below with reference to the accompanying drawings.

### Embodiment 1: Construction of the Genome-wide Insulator Screening System MAI-seq

### 1. Construction Method of the Genome-wide Insulator Screening System MAI-seq

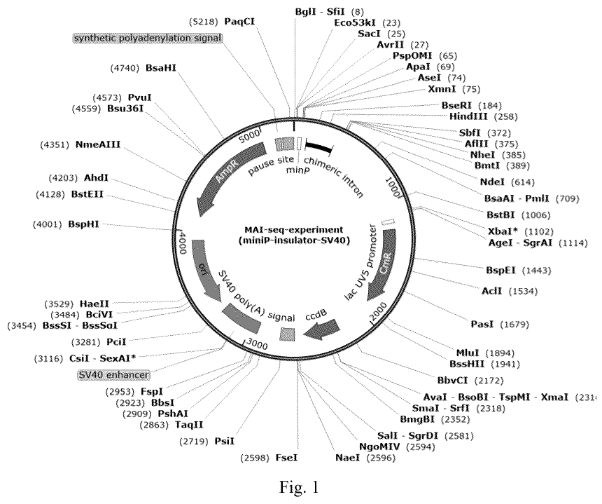

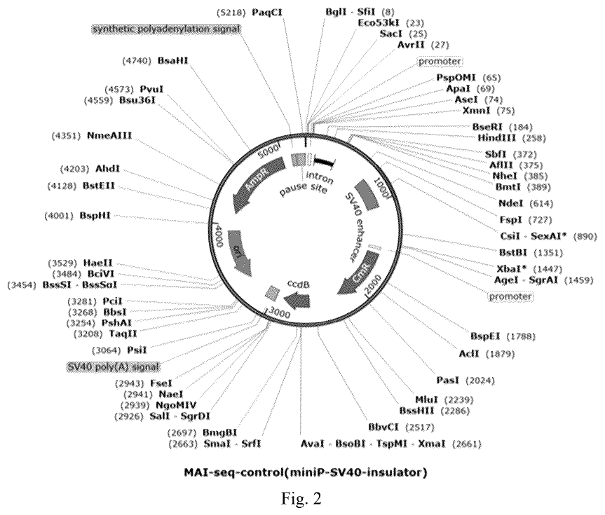

The genome-wide insulator screening system MAI-seq consists of two sets of vectors (a dual-vector system). The first vector is MAI-seq-experiment (as shown in FIG. 1), with its nucleotide sequence provided in SEQ ID NO:1. This vector is modified from the STARR-seq vector (addgene, 711509). Specifically, the SCP1 promoter of the STARR-seq vector is replaced with a Mini-promoter (a weak promoter containing an incomplete promoter sequence with a TATA-box, which requires interaction with an enhancer for transcription), and the SV40 enhancer sequence is inserted between the restriction enzyme sites BbsI and PciI. The second vector is MAI-seq-control (as shown in FIG. 2), with its nucleotide sequence provided in SEQ ID NO:2. This vector is also modified from the STARR-seq vector. Specifically, the SCP1 promoter of the STARR-seq vector is replaced with a Mini-promoter (a weak promoter), and the SV40 enhancer sequence is inserted at the BsaAI-PmlI site. The difference between the MAI-seq-experiment vector and the MAI-seq-control vector lies in the position of the SV40 enhancer, leading to distinct functions of the two.

### 2. Working Principle of the Genome-wide Insulator Screening System MAI-seq

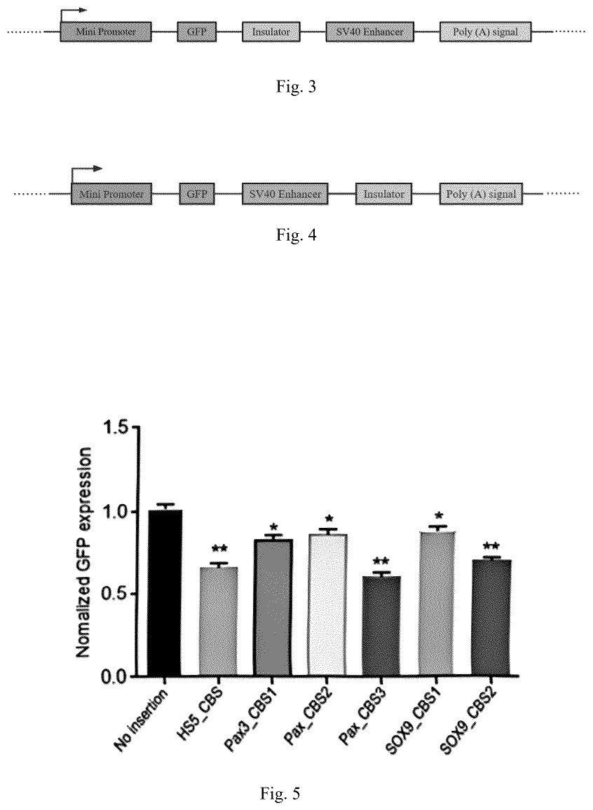

Insulators function in a position-dependent manner, only blocking the activation of target gene transcription by an enhancer when placed between the enhancer and the target gene. If the insulator is located elsewhere, it cannot prevent the enhancer from activating the target gene transcription. Therefore, the sequence to be screened is inserted between a “weak promoter, Mini Promoter” and an “enhancer, SV40 Enhancer.” In this way, sequences with insulator activity can block or attenuate the transcription of the target gene. The strength of each insulator can be reflected by the relative transcription abundance of the target gene within the cells. As shown in FIG. 3: When the sequence to be screened or identified (located at the “Insulator” position in FIG. 3) is an insulator, it will prevent the interaction between the “enhancer, SV40 Enhancer” and the “weak promoter, Mini Promoter,” thereby reducing the expression level of the marker gene GFP (the target gene). The transcription abundance and expression of the marker gene GFP will decrease. The strength of the insulator can be identified based on the degree of reduction in transcription abundance. Conversely, when the sequence to be screened or identified is not an insulator, the interaction between the “enhancer, SV40 Enhancer” and the “weak promoter, Mini Promoter” can activate the expression of the marker gene GFP, resulting in higher transcription abundance and expression levels of the marker gene GFP.

The reason for using the weak promoter, Mini Promoter, is that it can eliminate the influence of silencers to some extent. If a non-weak promoter (such as a strong promoter or a normal promoter) is used, when the sequence to be screened is a silencer, it may also lead to a decrease in the transcription abundance of the marker gene within the cells. In this case, some silencers may be mistakenly identified as insulators, resulting in false positives. Therefore, the two vectors of this system must use the interaction between a weak promoter and an enhancer to screen and identify insulators.

In the genome-wide insulator screening system MAI-seq, the core part of the MAI-seq experiment is shown in FIG. 3. Briefly, the sequence to be tested is inserted into the “Insulator” position in FIG. 3 through homologous recombination to obtain a homologous recombination plasmid. These plasmids are then pooled to construct an input library. Subsequently, the input library is transfected into cells, and cells are collected 24 hours later to construct an output library. For each sequence to be tested, if it possesses insulator activity, it can block the interaction between the Mini Promoter and the SV40 enhancer, thereby reducing the transcription abundance of the downstream target gene (GFP) sequence driven by the Mini Promoter. At the same time, the sequence to be tested will also be transcribed. By comparing the differences in transcription abundance of the sequence to be tested between the input and output libraries, it is possible to determine which sequences have insulator activity (if the sequence to be tested is an insulator, its transcription abundance in the output library will be lower than that in the input library).

Although the use of a weak promoter in the MAI-seq-experiment vector can avoid the influence of silencers on the screening results to some extent, to completely eliminate the impact of silencers, the system also includes the MAI-seq-control vector, whose core part is shown in FIG. 4. This is because insulators only exert their blocking effect when positioned between a promoter and an enhancer, whereas silencers can exert their blocking effect regardless of their position. Therefore, when the sequence to be tested is inserted into the “Insulator” position in FIG. 4, it will be selected and identified as having silencer activity only if it inhibits transcription. If the sequence to be tested is an insulator, it cannot inhibit transcription because it is not located between the promoter and the enhancer. Thus, the MAI-seq-control vector can be used to screen for silencer sequences.

By comparing the sequences screened using the MAI-seq-experiment and MAI-seq-control vectors, first list the sequences screened by the MAI-seq-experiment vector, and then list the sequences screened by the MAI-seq-control vector. Then identify the overlapping sequences between the two sets of screened sequences. The silencer sequences that are found in both sets (i.e., the overlapping part) are removed from the sequences screened by the MAI-seq-experiment vector. Ultimately, the remaining sequences are identified as insulator sequences.

In summary, the insulators ultimately identified by the MAI-seq system are: the sequences screened and identified by the MAI-seq-experiment vector (insulators+silencers) minus the overlapping sequences screened and identified by both the MAI-seq-experiment and MAI-seq-control vectors (silencers).

Furthermore, in the genome-wide insulator screening system MAI-seq, the enhancer can be not only the SV40 Enhancer but also other enhancers such as the CMV Enhancer, etc. The marker target gene can be not only the GFP gene but also other genes such as RFP, mCherry, BFP, etc.

### Embodiment 2: Functional Validation of the MAI-seq-experiment Vector in the Genome-wide Insulator Screening System MAI-seq

To validate whether MAI-seq can screen and identify insulator sequences, the sequences of previously reported insulators (Table 1) were cloned into the MAI-seq-experiment vector. These included a control group (No Insulator) and experimental groups with the sequences HS5_CBS, Pax3_CBS1, Pax3_CBS2, Pax3_CBS3, SOX9_CBS1, and SOX9_CBS2 from Table 1. The control and experimental groups were transfected into cells, and after 48 hours, the cells were collected and RNA was extracted. qPCR was used to detect changes in GFP gene expression (detection primers are shown in Table 2).

The results are shown in FIG. 5: compared to the control group, GFP expression in the experimental groups was significantly or extremely significantly reduced (in FIG. 5, ** indicates a highly significant difference compared to the control group, p<0.01; * indicates a significant difference compared to the control group, p<0.05). These results demonstrate that the reported insulator sequences in Table 1 can be efficiently detected using the MAI-seq-experiment vector, indicating that the MAI-seq-experiment vector can be used for the screening or identification of insulators.

TABLE 1
Information on Reported Insulators Used
to Validate the MAI-seq-experiment Vector

| | | | | Reference |
|---|---|---|---|---|
| Sequence | Chromatin | Start Position | End Position | Genome |
| HS5_CBS | Chr11 | 5310702 | 5313343 | hg19 |
| Pax3_CBS1 | Chr1 | 77942190 | 77944770 | mm10 |
| Pax3_CBS2 | chr1 | 77970975 | 77974415 | mm10 |
| Pax3_CBS3 | Chr1 | 77976869 | 77980896 | mm10 |
| SOX9_CBS1 | chr11 | 111536561 | 111538959 | mm10 |
| SOX9_CBS2 | Chr11 | 111533965 | 111536560 | mm10 |

TABLE 2
qPCR Detection Primer Sequences

| Primer Name | Sequence |
|---|---|
| GFP-F primer | 5′-ACCCTGAAGTTCATCTGCAC-3′ |
| | (SEQ ID NO: 3) |
| GFP-R primer | 5′-CATGCCGTTTCATATGATCC-3′ |
| | (SEQ ID NO: 4) |

### Embodiment 3: Genome-wide Insulator Screening Method

### 3.1 Preparation of Input Library

Cultivate the target cells in which insulators need to be screened. When the cell confluence reaches 80%-90%, collect the cells and extract genomic DNA. Fragment the DNA into approximately 700 bp segments using an ultrasonic device, and then perform end-repair on the DNA fragments. Use the NEB® (NEB®; cat. no. E6000L) DNA Library Prep Kit to ligate Illumina® sequencing adapters (Illumina Inc®; cat. no. PE-400-1001) to 5 μg of the DNA fragments. Amplify the DNA using KAPA® amplification enzyme (KAPA Biosystems®; cat. no. KK2602) and primers containing vector homology arms. The primer sequences are as follows:

fw (SEQ ID NO: 5):
TAGAGCATGCACCGGACACTCTTTCCCTACACGACGCTCTTCCGAT
CT;
rev (SEQ ID NO: 6):
GGCCGAATTCGTCGAGTGACTGGAGTTCAGACGTGTGCTCTTCCG
ATCT.

The fw (SEQ ID NO:5) and rev (SEQ ID NO:6) primers contain homology arm sequences that can undergo homologous recombination with both the MAI-seq-experiment vector and the MAI-seq-control vector. The DNA fragments amplified using these primers will also have homology arm sequences at both ends that can recombine with the MAI-seq-experiment and MAI-seq-control vectors. Through homologous recombination, the DNA fragments to be screened/tested can be inserted into the designated position of the vector (i.e., the insertion site for the sequence to be screened, as shown in FIG. 3 or 4 under “Insulator”).

Next, PCR amplification is performed according to the set program (98° C. for 45 s; 98° C. for 15 s, 65° C. for 30 s, 72° C. for 30 s for 10 cycles). The amplification products are purified using magnetic beads (ratio of beads/PCR 0.8; cat. no. A63881) to obtain the constructed DNA fragments. The MAI-seq-experiment and MAI-seq-control vectors are linearized using the restriction enzymes SalI-HF and AgeI-HF. The enzyme-digested products are subjected to agarose gel electrophoresis, and the desired products are recovered to obtain the linearized vectors. Following the instructions of the homologous recombination kit (Nanjing Vazyme®, C117), the constructed DNA fragments are subjected to homologous recombination with the linearized MAI-seq-experiment and MAI-seq-control vectors, respectively, to construct homologous recombination plasmids. The homologous recombination plasmids are transformed into electrocompetent cells, and all transformation products are combined and placed in LB medium. The culture is shaken on a shaker at 37° C. and 200 rpm until the OD value of the bacterial suspension reaches 0.8-1.2. The plasmids are extracted using a kit (OMEGA®, D6902), and all plasmids are combined to form the Input library, which is then subjected to high-throughput sequencing.

### 3.2 Construction of Output Library

The constructed MAI-seq-experiment and MAI-seq-control input libraries are separately transfected into the target cells. After 24 hours of transfection, the transfected cells are collected separately. Total RNA is extracted from the collected cells using the M5 Universal Plus RNA Mini Kit, and mRNA with a poly (A) tail is enriched using the Dynabeads Oligo (dT) kit (Thermo Fisher Scientific®, 61005), which contains Oligo dT-coated magnetic beads.

Subsequently, the enriched mRNA is treated with TURBO™ DNase (Thermo Fisher Scientific®) to remove genomic DNA, and then purified using Agencourt RNA Clean XP beads (Beckman Coulter®). The mRNA is specifically reverse transcribed into target-specific first-strand complementary DNA (cDNA) using SuperScript III reverse transcriptase (Thermo Fisher Scientific®, 1.5 μg mRNA per reaction). The reverse transcription primer used is 5′-CAAACTCATCAATGTATCTTATCATG-3′ (SEQ ID NO: 7).

The cDNA is treated with RNase A+H and then purified using a PCR & DNA Clean up Kit. Finally, using this cDNA as a template, PCR amplification is performed with the following primers:

The forward primer F is
(SEQ ID NO: 8)
5′-AATGATACGGCGACCACCGAGATCTACACTCTTTCCCTACACGACGC
TCTTCCGATCT-3′;
The reverse primer R is
(SEQ ID NO: 9)
5′-CAAGCAGAAGACGGCATACGAGAT-index-
GTGACTGGAGTTCAGACGTG-3′.

Each 50 μL PCR reaction mixture contains 5 μL of cDNA, and PCR amplification is performed using KAPAR amplification enzyme (KAPA Biosystems®; cat. no. KK2602) with the following program: 98° C. for 30 s for initial denaturation; 98° C. for 10 s for denaturation, 65° C. for 30 s for annealing, and 72° C. for 30 s for extension, for a total of 20 cycles. The amplified PCR products are subjected to agarose gel electrophoresis, and the target bands are recovered using a gel recovery kit to prepare the output libraries for the MAI-seq-experiment and MAI-seq-control vectors, respectively, which are then subjected to high-throughput sequencing.

The MAI-seq system (comprising MAI-seq-experiment and MAI-seq-control vectors) is used for homologous recombination with the sequence to be screened to obtain both the Input and Output libraries. These two libraries consist of two parts of data with different read types. The Input library is constructed by directly amplifying the insert fragments from the plasmid DNA used for cell transfection, serving as a reference for the original representation of insert fragments in the starting plasmid mixture. The Output library, on the other hand, is generated by measuring the abundance of mRNA transcribed from the insert fragments in the transfected plasmid pool.

### 3.3 Identification of Insulators

After obtaining the input and output sequencing data for the MAI-seq-experiment and MAI-seq-control vectors, the CRADLE software is used to process the data. To enhance the reliability of the final identified insulators, the following specific steps are taken:

First, only sequences with more than 20 reads in both the input and output libraries are selected for analysis.

Second, the BAM files for the input and output of both vectors are converted to BW files.

Third, the correctBias function is used to correct for technical biases in the read counts (shearing, PCR, mappability, G-quadruplex).

Fourth, the call peaks command is used to identify sequences with repressive effects. The key parameters are as follows: -rbin 300, -wbin 100, -d 20, -fdr 0.05.

Through these steps, sequences with reduced transcriptional abundance in the output library compared to the input library are selected. Then, the sequencing data from the input and output libraries of the MAI-seq-control vector are compared, and sequences with reduced transcriptional abundance in the output library compared to the input library are also selected. Subsequently, the sequences identified from both the MAI-seq-experiment and MAI-seq-control vectors are compared for overlapping parts. The sequences from the MAI-seq-experiment vector are used to remove the overlapping sequences, and the remaining sequences are identified as insulator sequences.

In summary, the final insulator sequences are those identified from the MAI-seq-experiment vector, with the overlapping sequences removed that were also identified from both the MAI-seq-experiment and MAI-seq-control vectors. The remaining sequences are the insulator sequences.

### 3.4 Validation of the MAI-seq System

To validate the function of the MAI-seq system in screening for insulators, 30 sequences were synthesized, including 5 known insulator sequences, 5 known silencer sequences, and 20 negative sequences. Following the procedures outlined in sections 3.1 and 3.2 of Embodiment 3, the input and output libraries for the MAI-seq-experiment and MAI-seq-control vectors were constructed, and sequence identification was performed. The results showed that the MAI-seq-experiment vector system identified 10 sequences, including 5 silencers and 5 insulators, while the MAI-seq-control identified 5 silencer sequences. The overlapping sequences identified by both vectors were 5 silencer sequences, so the final sequences were 5 (5=10-5) insulator sequences, which was consistent with expectations. The validation results are shown in Table 3, demonstrating the efficiency and reliability of the MAI-seq system.

TABLE 3
Validation Results of the MAI-seq System

| | | | Negative |
|---|---|---|---|
| Total Tested Sequence Types | Insulators | Silencers | Sequences |
| Total Tested Sequence Count | 5 | 5 | 20 |
| Number of Sequences Screened | 5 | 5 | 0 |
| by MAI-seq-experiment Vector | | | |
| Number of Sequences Screened | 0 | 5 | 0 |
| by MAI-seq-control Vector | | | |
| Final Number of Insulators | 5 | 0 | 0 |

1. A genome-wide insulator screening system (MAI-seg), wherein the system comprises an MAI-seq-experiment vector and an MAI-seq-control vector; core gene elements in the MAI-seq-experiment vector are arranged in the following order: a weak promoter, a marker target gene, an insertion site for a sequence to be screened, an enhancer and a poly A site; core gene elements in the MAI-seq-control vector are arranged in the following order: a weak promoter, a marker target gene, an enhancer, an insertion site for the sequence to be screened and a poly A site; the insertion site for the sequence to be screened contains homologous arm sequences that are configured to hybridize with complementary sequences attached to the DNA sequence to be screened to enable homologous recombination, and after two ends of a DNA sequence to be screened are added to the homologous arm sequences, homologous recombination occurs with either the MAI-seq-experiment vector or the MAI-seq-control vector; and a nucleotide sequence of the MAI-seq-experiment vector is as shown in SEQ ID NO:1, and a nucleotide sequence of the MAI-seq-control vector is as shown in SEQ ID NO:2.

2. A method for screening for genomic insulators using the genome-wide insulator screening system according to claim 1, comprising following steps:
S1: fragmenting a gene sequence to be tested, ligating the fragmented gene segments with sequencing adapters, and performing PCR amplification using primers as shown in SEQ ID NO:5 and SEQ ID NO:6;
S2: homologously recombining PCR products from S1 into the MAI-seq-experiment vector and the MAI-seg-control vector according to claim 1 to construct homologous recombination plasmids;
S3: transforming the homologous recombination plasmids constructed in S2 and extracting the plasmids to obtain an Input library for the MAI-seq-experiment vector and an Input library for the MAI-seg-control vector, respectively, and performing high-throughput sequencing on the Input libraries;
S4: transfecting cells with the Input libraries of the MAI-seq-experiment vector and the MAI-seq-control vector constructed in S3, collecting the cells, performing reverse transcription amplification, collecting amplified products to prepare an Output library for the MAI-seg-experiment vector and an Output library for the MAI-seg-control vector, respectively, and performing high-throughput sequencing on the Output libraries;
S5: by comparing sequencing data of the Input and Output libraries of the MAI-seg-experiment vector, screening out sequences with reduced transcription abundance in the Output library compared to the Input library;
S6: by comparing sequencing data of the Input and Output libraries of the MAI-seq-control vector, screening out sequences with reduced transcription abundance in the Output library compared to the Input library; and
S7: removing from the sequences screened out in S5 those that are duplicated in the sequences screened out in S6, and defining sequences obtained after the removing as the screened insulator sequences.

3. (canceled)

4. A method for validating genomic insulators using the genome-wide insulator screening system according to claim 1, comprising following steps:
S1: inserting the sequence to be validated into the MAI-seq-experiment vector to construct a validation plasmid;
S2: using the MAI-seq-experiment vector without any sequence inserted as a blank control plasmid; and
S3: transfecting cells with the blank control plasmid and the validation plasmid separately, collecting cells 48 hours post-transfection to extract RNA, and utilizing qPCR to detect changes in the expression level of the marker target gene; and identifying the sequence to be validated is an insulator if the expression level of the marker target gene in the validation plasmid group decreases.

5. (canceled)

6. (canceled)

7. An insulator validation vector comprising the nucleotide sequence set forth in SEQ ID NO: 1.


SEQUENCE LISTING (9 sequences)

SEQ ID NO: 1 - DNA, 5248 bp, synthetic construct
  source 1..5248: mol_type = other DNA
ggcctaactggccggtacctgagctccctaggtagagggtatataatggaagctcgacttccaggggcccgaattaattcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcgcctgcaggcttaagcatggctagcaaaggagaagaactcttcactggagttgtcccaattcttgttgaattagatggtgatgttaacggccacaagttctctgtcagtggagagggtgaaggtgatgcaacatacggaaaacttaccctgaagttcatctgcactactggcaaactgcctgttccctggccaacactagtcactactctgtgctatggtgttcaatgcttttcaagatacccggatcatatgaaacggcatgactttttcaagagtgccatgcccgaaggttatgtacaggaaaggaccatcttcttcaaagatgacggcaactacaagacacgtgctgaagtcaagtttgaaggtgatacccttgttaatagaatcgagttaaaaggtattgacttcaaggaagatggcaacattctgggacacaaattggaatacaactataactcacacaatgtatacatcatggcagacaaacaaaagaatggaatcaaagcgaacttcaagacccgccacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactgtacaactgatctagagcatgcaccggtgatatcgcggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggatatcgtcgacgaattcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgaccgatgcccttgagagccttcaacccagtcagctccttccggtgggcgcggggcatgactatcgtcgccgcacttatgactgtcttccgaggatatcagatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttattacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagcggccgcaaatgctaaaccactgcagtggttaccagtgcttgatcagtgaggcaccgatctcagcgatctgcctatttcgttcgtccatagtggcctgactccccgtcgtgtagatcactacgattcgtgagggcttaccatcaggccccagcgcagcaatgatgccgcgagagccgcgttcaccggcccccgatttgtcagcaatgaaccagccagcagggagggccgagcgaagaagtggtcctgctactttgtccgcctccatccagtctatgagctgctgtcgtgatgctagagtaagaagttcgccagtgagtagtttccgaagagttgtggccattgctactggcatcgtggtatcacgctcgtcgttcggtatggcttcgttcaactctggttcccagcggtcaagccgggtcacatgatcacccatattatgaagaaatgcagtcagctccttagggcctccgatcgttgtcagaagtaagttggccgcggtgttgtcgctcatggtaatggcagcactacacaattctcttaccgtcatgccatccgtaagatgcttttccgtgaccggcgagtactcaaccaagtcgttttgtgagtagtgtatacggcgaccaagctgctcttgcccggcgtctatacgggacaacaccgcgccacatagcagtactttgaaagtgctcatcatcgggaatcgttcttcggggcggaaagactcaaggatcttgccgctattgagatccagttcgatatagcccactcttgcacccagttgatcttcagcatcttttactttcaccagcgtttcggggtgtgcaaaaacaggcaagcaaaatgccgcaaagaagggaatgagtgcgacacgaaaatgttggatgctcatactcgtcctttttcaatattattgaagcatttatcagggttactagtacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctct

SEQ ID NO: 2 - DNA, 5248 bp, synthetic construct
  source 1..5248: mol_type = other DNA
ggcctaactggccggtacctgagctccctaggtagagggtatataatggaagctcgacttccaggggcccgaattaattcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcgcctgcaggcttaagcatggctagcaaaggagaagaactcttcactggagttgtcccaattcttgttgaattagatggtgatgttaacggccacaagttctctgtcagtggagagggtgaaggtgatgcaacatacggaaaacttaccctgaagttcatctgcactactggcaaactgcctgttccctggccaacactagtcactactctgtgctatggtgttcaatgcttttcaagatacccggatcatatgaaacggcatgactttttcaagagtgccatgcccgaaggttatgtacaggaaaggaccatcttcttcaaagatgacggcaactacaagacaccgaggatatcagatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttattgtgctgaagtcaagtttgaaggtgatacccttgttaatagaatcgagttaaaaggtattgacttcaaggaagatggcaacattctgggacacaaattggaatacaactataactcacacaatgtatacatcatggcagacaaacaaaagaatggaatcaaagcgaacttcaagacccgccacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactgtacaactgatctagagcatgcaccggtgatatcgcggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggatatcgtcgacgaattcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgaccgatgcccttgagagccttcaacccagtcagctccttccggtgggcgcggggcatgactatcgtcgccgcacttatgactgtcttcacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagcggccgcaaatgctaaaccactgcagtggttaccagtgcttgatcagtgaggcaccgatctcagcgatctgcctatttcgttcgtccatagtggcctgactccccgtcgtgtagatcactacgattcgtgagggcttaccatcaggccccagcgcagcaatgatgccgcgagagccgcgttcaccggcccccgatttgtcagcaatgaaccagccagcagggagggccgagcgaagaagtggtcctgctactttgtccgcctccatccagtctatgagctgctgtcgtgatgctagagtaagaagttcgccagtgagtagtttccgaagagttgtggccattgctactggcatcgtggtatcacgctcgtcgttcggtatggcttcgttcaactctggttcccagcggtcaagccgggtcacatgatcacccatattatgaagaaatgcagtcagctccttagggcctccgatcgttgtcagaagtaagttggccgcggtgttgtcgctcatggtaatggcagcactacacaattctcttaccgtcatgccatccgtaagatgcttttccgtgaccggcgagtactcaaccaagtcgttttgtgagtagtgtatacggcgaccaagctgctcttgcccggcgtctatacgggacaacaccgcgccacatagcagtactttgaaagtgctcatcatcgggaatcgttcttcggggcggaaagactcaaggatcttgccgctattgagatccagttcgatatagcccactcttgcacccagttgatcttcagcatcttttactttcaccagcgtttcggggtgtgcaaaaacaggcaagcaaaatgccgcaaagaagggaatgagtgcgacacgaaaatgttggatgctcatactcgtcctttttcaatattattgaagcatttatcagggttactagtacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctct

SEQ ID NO: 3 - DNA, 20 bp, synthetic construct
  source 1..20: mol_type = other DNA
accctgaagttcatctgcac

SEQ ID NO: 4 - DNA, 20 bp, synthetic construct
  source 1..20: mol_type = other DNA
catgccgtttcatatgatcc

SEQ ID NO: 5 - DNA, 48 bp, synthetic construct
  source 1..48: mol_type = other DNA
tagagcatgcaccggacactctttccctacacgacgctcttccgatct

SEQ ID NO: 6 - DNA, 49 bp, synthetic construct
  source 1..49: mol_type = other DNA
ggccgaattcgtcgagtgactggagttcagacgtgtgctcttccgatct

SEQ ID NO: 7 - DNA, 26 bp, synthetic construct
  source 1..26: mol_type = other DNA
caaactcatcaatgtatcttatcatg

SEQ ID NO: 8 - DNA, 58 bp, synthetic construct
  source 1..58: mol_type = other DNA
aatgatacggcgaccaccgagatctacactctttccctacacgacgctcttccgatct

SEQ ID NO: 9 - DNA, 44 bp, synthetic construct
  source 1..44: mol_type = other DNA
caagcagaagacggcatacgagatgtgactggagttcagacgtg